(19) United States
(12) Patent Application Publication
(10) Pub. No.: US 2014/0309460 A1
(43) Pub. Date: Oct. 16, 2014

(54) INCREASING THE MOLAR MASS OF POLYALKYLENEPOLYAMINES BY HOMOGENEOUSLY CATALYZED ALCOHOL AMINATION

(71) Applicant: BASF SE, Ludwigshafen (DE)

(72) Inventors: Julia Strautmann, Mannheim (DE); Thomas Schaub, Neustadt (DE); Stephan Hueffer, Ludwigshafen (DE); Rocco Paciello, Bad Duerkheim (DE)

(73) Assignee: BASF SE, Ludwigshafen (DE)

(21) Appl. No.: 14/357,822

(22) PCT Filed: Nov. 19, 2012
(86) PCT No.: PCT/EP12/72942

§ 371 Date: May 13, 2014

(30) Foreign Application Priority Data
Nov. 25, 2011 (EP) 11190837.2

(51) Int. Cl. C07C 209/16; C07C 211/02

(52) U.S. Cl. CPC C07C 209/16; C07C 211/02
USPC 564/480; 564/512

(57) ABSTRACT

Process for increasing the molar mass of polyalkylenepolyamines by homogeneously catalyzed alcohol amination, which comprises carrying out a reaction of the polyalkylenepolyamines in a reactor with elimination of water in the presence of a homogeneous catalyst and removing the water of reaction from the reaction system. Polyalkylenepolyamines obtainable by such processes, and polyalkylenepolyamines comprising hydroxyl groups, secondary amines or tertiary amines. Uses of such polyalkylenepolyamines as adhesion promoters for printing inks, adhesion promoters in composite films, cohesion promoters for adhesives, crosslinkers/curing agents for resins, primers for paints, wet-adhesion promoters for emulsion paints, complexing agents and flocculating agents, penetration assistants in wood preservation, corrosion inhibitors, immobilizing agents for proteins and enzymes.

The present invention relates to processes for increasing the molar mass of polyalkylenepolyamines by homogeneously catalyzed alcohol amination. Furthermore, the invention also relates to polyalkylenepolyamines obtainable by these processes and to the use of polyalkylenepolyamines. The invention further provides specific polyalkylenepolyamines having hydroxyl groups, secondary amine groups or tertiary amine groups.

Further embodiments of the present invention can be found in the claims, the description and the examples. It goes without saying that the features of the subject matter according to the invention that have been specified above and are still to be explained below can be used not only in the combination specifically stated in each case, but also in other combinations, without departing from the scope of the invention. The embodiments of the present invention in which all features have the preferred or very preferred meanings are preferred or very preferred, respectively.

Polyethyleneimines are valuable products with a large number of different uses. For example, polyethyleneimines are used: a) as adhesion promoters for printing inks for laminate films; b) as auxiliaries (adhesion) for producing multi-ply composite films, where not only are different polymer layers compatibilized, but also metal films; c) as adhesion promoters for adhesives, for example in conjunction with polyvinyl alcohol, butyrate and acetate and styrene copolymers, or as cohesion promoters for label adhesives; d) low molecular weight polyethyleneimines can moreover be used as crosslinkers/curing agents in epoxy resins and polyurethane adhesives; e) as primers in coating applications for improving adhesion on substrates such as glass, wood, plastic and metal; f) for improving wet adhesion in standard emulsion paints and also for improving the instantaneous rain resistance of paints for example for road markings; g) as complexing agent with high binding capacity for heavy metals such as Hg, Pb, Cu, Ni and flocculating agents in water treatment/water processing; h) as penetration assistants for active metal salt formulations in wood preservation; i) as corrosion inhibitors for iron and nonferrous metals; j) for the immobilization of proteins and enzymes. For these applications, it is also possible to use polyalkylenepolyamines which are not derived from the ethyleneimine.

Polyethyleneimines are currently obtained by the homopolymerization of ethyleneimine. Ethyleneimine is a highly reactive, corrosive and toxic intermediate which can be synthesized in different ways (aziridines, Ulrich Steuerle, Robert Feuerhake; in Ullmann's Encyclopedia of Industrial Chemistry, 2006, Wiley-VCH, Weinheim).

For the preparation of polyalkylenepolyamines —[(CH₂)ₓN]— with alkylene groups >C₂ (x>2) not derived from aziridine, there are no processes analogous to the aziridine route, as a result of which there has hitherto been no cost-effective process for their preparation.

The homogenously catalyzed amination of alcohols is known from the literature for the synthesis of primary, secondary and tertiary amines starting from alcohols and amines, with monomeric products being obtained in all of the described embodiments.

U.S. Pat. No. 3,708,539 describes the synthesis of primary, secondary and tertiary amines using a ruthenium-phosphane complex. Y. Watanabe, Y. Tsuji, Y. Ohsugi Tetrahedron Lett. 1981, 22, 2667-2670 reports on the preparation of arylamines by the amination of alcohols with aniline using [Ru(PPh₃)₃Cl₂] as catalyst.

EP 0 034 480 A2 discloses the preparation of N-alkyl- or N,N-dialkylamines by the reaction of primary or secondary amines with a primary or secondary alcohol using an iridium, rhodium, ruthenium, osmium, platinum, palladium or rhenium catalyst.

EP 0 239 934 A1 describes the synthesis of mono- and diaminated products starting from diols such as ethylene glycol and 1,3-propanediol with secondary amines using ruthenium and iridium phosphane complexes.

K. I. Fujita, R. Yamaguchi Synlett, 2005, 4, 560-571 describes the synthesis of secondary amines by the reaction of alcohols with primary amines and also the synthesis of cyclic amines by the reaction of primary amines with diols by ring closure using iridium catalysts.

In A. Tillack, D. Hollmann, K. Mevius, D. Michalik, S. Barn, M. Beller Eur. J. Org. Chem. 2008, 4745-4750, in A. Tillack, D. Hollmann, D. Michalik, M. Beller Tetrahedron Lett. 2006, 47, 8881-8885, in D. Hollmann, S. Bahn, A. Tillack, M. Beller Angew. Chem. Int. Ed. 2007, 46, 8291-8294 and in M. Haniti, S. A. Hamid, C. L. Allen, G. W. Lamb, A. C. Maxwell, H. C. Maytum, A. J. A. Watson, J. M. J. Williams J. Am. Chem. Soc, 2009, 131, 1766-1774 syntheses of secondary and tertiary amines starting from alcohols and primary or secondary amines using homogeneous ruthenium catalysts are described.

The synthesis of primary amines by reacting alcohols with ammonia using a homogeneous ruthenium catalyst is reported in C. Gunanathan, D. Milstein Angew. Chem. Int. Ed. 2008, 47, 8661-8664.

Our unpublished application PCT/EP2011/058758 describes general processes for the preparation of polyalkylenepolyamines by catalytic alcohol amination of alkanolamines or of diamines or polyamines with diols or polyols.

It was an object of the present invention to find a process for increasing the molar mass of polyalkylenepolyamines in which no aziridine is used, no undesired coproducts are formed and products of a desired chain length are obtained. A further object was to provide processes which make it possible, starting from existing polyalkylenepolyamine reactants, to obtain polyalkylenepolyamines having a higher molar mass in comparison to these polyalkylenepolyamine reactants.

These and other objects are achieved, as is evident from the disclosure content of the present invention, by the various embodiments of the process of the invention for increasing the molar mass of polyalkylenepolyamines by catalyzed alcohol amination, in which a reaction of the polyalkylenepolyamines is carried out in a reactor with elimination of water in the presence of a homogeneous catalyst, and the water of reaction is removed from the reaction system.

By water of reaction is meant the water formed in the elimination of water during the reaction of hydroxyl groups and amino groups of the monomers.

By room temperature is meant 21° C.

Within the context of this invention, expressions of the form Cₐ-C_{b} refer to chemical compounds or substituents with a certain number of carbon atoms. The number of carbon atoms can be selected from the entire range from a to b, including a and b, a is at least 1 and b is always greater than a. The chemical compounds or substituents are further specified by expressions of the form Cₐ-C_{b}-V. V here stands for a chemical compound class or substituent class, for example alkyl compounds or alkyl substituents.

Specifically, the collective terms stated for the various substituents have the following meaning:

C₁-C₅₀-Alkyl: straight-chain or branched hydrocarbon radicals having up to 50 carbon atoms, for example C₁-C₁₀-alkyl or C₁₁-C₂₀-alkyl, preferably C₁-C₁₀-alkyl, for example C₁-C₃-alkyl, such as methyl, ethyl, propyl, isopropyl, or C4-C6-alkyl, n-butyl, sec-butyl, tert-butyl, 1,1-dimethylethyl, pentyl, 2-methylbutyl, 1,1-dimethylpropyl, 1,2-dimethylpropyl, 2,2-dimethylpropyl, 1-ethylpropyl, hexyl, 2-methylpentyl, 3-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 2-ethylbutyl, 1,1,2-trimethylpropyl, 1,2,2-trimethylpropyl, 1-ethyl-1-methylpropyl, 1-ethyl-2-methylpropyl, or C₇-C₁₀-alkyl, such as heptyl, octyl, 2-ethylhexyl, 2,4,4-trimethylpentyl, 1,1,3,3-tetramethylbutyl, nonyl or decyl, and isomers thereof.

C₃-C₁₅-Cycloalkyl: monocyclic, saturated hydrocarbon groups having from 3 up to 15 carbon ring members, preferably C₃-C₈-cycloalkyl such as cyclopropyl, cyclobutyl, cyclopentyl, cyclohexyl, cycloheptyl or cyclooctyl, and also a saturated or unsaturated cyclic system such as e.g. norbornyl or norbenyl.

Aryl: a mono- to trinuclear aromatic ring system comprising 6 to 14 carbon ring members, e.g. phenyl, naphthyl or anthracenyl, preferably a mono- to dinuclear, particularly preferably a mononuclear, aromatic ring system.

Within the context of the present invention, the symbol “*” indicates, for all chemical compounds, the valence via which one chemical group is bonded to another chemical group.

Polyalkylenepolyamines can be obtained, for example, by reacting (i) aliphatic amino alcohols with one another, with elimination of water, or by reacting (ii) aliphatic diamines or polyamines with aliphatic dioles or polyols, with elimination of water, in each case in the presence of a catalyst. Processes of these kinds are described in our unpublished application PCT/EP2011/058758, for example.

In a first preferred embodiment of the process of the invention for increasing the molar mass, the water of reaction is removed during such a reaction or preparation of polyalkylenepolyamines by homogeneously catalyzed alcohol amination. This means that, during the operation for preparing the polyalkylenepolyamines by reaction of (i) aliphatic amino alcohols with one another, with elimination of water, or of (ii) aliphatic diamines or polyamines with aliphatic dioles or polyols with eliminatin of water, in each case in the present of a homogeneous catalyst, the water of reaction is removed. An additional removal of the water of reaction may also take place here after the preparation of the polyalkylenepolyamines.

In a second preferred embodiment (first postcrosslinking mode) of the process of the invention for increasing the molar mass, polyalkylenepolyamines of relatively low molar mass are used, which have been prepared by any desired processes, examples being those mentioned above. These polyalkylenepolyamines of relatively low molar mass can be used directly after their preparation or, optionally, following isolation and/or purification, preferably after the removal of existing water as starting materials for the preparation of polyalkylenepolyamines of higher molar mass. In accordance with the invention, the molar mass of the polyalkylenepolyamines of relatively low molar mass is increased as part of a first postcrosslinking mode, by the polyalkylenepolyamines of relatively low molar mass being reacted in the presence of a homogeneous catalyst, with elimination of water, and the water of reaction being stripped from the system. In this case the polyalkylenepolyamines of relatively low molar mass preferably comprise free hydroxyl groups and amino groups, in order to allow the first postcrosslinking mode by alcohol amination. Preferably, furthermore, after the preparation of the polyalkylenepolyamines of relatively high molar mass, water present is removed. In one preferred embodiment the sequence composed of a) reacting the polyalkylenepolyamine of relatively low molar mass in the presence of a catalyst, and b) removing the water of reaction, is repeated up to 30 times, with the molar mass of the polyalkylenepolyamine of relatively high molar mass increasing in each step sequence.

It is of course possible to combine the first and second preferred embodiments of the process of the invention, in order to ensure a further increase in the molar mass.

In a third preferred embodiment of the process of the invention, a so-called second postcrosslinking mode is carried out for the purpose of increasing the molar mass. In the case of this second postcrosslinking mode, in the context of the present invention, in a first step, polyalkylenepolyamines of relatively low molar mass are provided, having been prepared by any desired processes—for example, the processes described above. These polyalkylenepolyamines of relatively low molar mass, directly after their preparation or, optionally, after isolation and/or purification, preferably after removal of existing water, can be used as starting materials. In a second step, the second postcrosslinking mode is carried out, wherein a polyalkylenepolyamine of relatively low molar mass and (i) aliphatic amino alcohols or (ii) aliphatic diamines or polyamines with aliphatic dioles or polyols are added. Here, the polyalkylenepolyamine of relatively molar mass and (i) aliphatic amino alcohols or (ii) aliphatic diamines or polyamines with aliphatic dioles or polyols are used as reactants, and are reacted with elimination of water and removal of the water of reaction from the reaction system, in the presence of a homogeneous catalyst, to give a polyalkylenepolyamine of relatively high molar mass. Here again, there may be an additional removal of the water of reaction after the polyalkylenepolyamines have been prepared. In one preferred embodiment the sequence composed of a) reaction of the polyalkylenepolyamine in the presence of a homogeneous catalyst and i) aliphatic amino alcohols or (ii) aliphatic diamines or polyamines with aliphatic dioles or polyols, and b) removal of the water of reaction, is repeated up to 30 times, with the molar mass of the polyalkylenepolyamine of relatively high molar mass increasing in each step sequence. As aliphatic diamine (ii) here it is preferred to use ethylenediamine.

Of course, it is possible to combine the first, second, and third preferred embodiments of the process of the invention, in order to ensure a further increase in the molar mass. Preferably it is possible, optionally after application of the first preferred embodiment, to combine the second and third preferred embodiments of the process of the invention one or more times in succession or in alternation, in order to ensure a further increase in the molar mass.

For increasing the molar mass it is possible in the context of the process of the invention to strip the water from the reaction system continuously during the reaction.

Aliphatic amino alcohols which are suitable for crosslinking of the second mode comprise at least one primary or secondary amino group and at least one OH group. Examples are linear, branched or cyclic alkanolamines such as monoethanolamine, diethanolamine, aminopropanol, for example 3-aminopropan-1-ol or 2-aminopropan-1-ol, aminobutanol, for example 4-aminobutan-1-ol, 2-aminobutan-1-ol or 3-aminobutan-1-ol, aminopentanol, for example 5-aminopentan-1-ol or 1-aminopentan-2-ol, aminodimethylpentanol, for example 5-amino-2,2-dimethylpentanol, aminohexanol, for example 2-aminohexan-1-ol or 6-aminohexan-1-ol, aminoheptanol, for example 2-aminoheptan-1-ol or 7-aminoheptan-1-ol, aminooctanol, for example 2-aminooctan-1-ol or 8-aminooctan-1-ol, aminononanol, for example 2-aminononan-1-ol or 9-aminononan-1-ol, aminodecanol, for example 2-aminodecan-1-ol or 10-aminodecan-1-ol, aminoundecanol, for example 2-aminoundecan-1-ol or 11-aminoundecan-1-ol, aminododecanol, for example 2-aminododecan-1-ol or 12-aminododecan-1-ol, aminotridecanol, for example 2-aminotridecan-1-ol, 1-(2-hydroxyethyl)piperazine, 2-(2-aminoethoxy)ethanol, alkylalkanolamines, for example butylethanolamine, propylethanolamine, ethylethanolamine, methylethanolamine.

Aliphatic diamines which are suitable for crosslinking of the second mode comprise at least two primary or at least one primary and one secondary or at least two secondary amino groups, they preferably comprise two primary amino groups. Examples are linear, branched or cyclic aliphatic diamines. Examples are ethylenediamine, 1,3-propylenediamine, 1,2-propylenediamine, butylenediamine, for example 1,4-butylenediamine or 1,2-butylenediamine, diaminopentane, for example 1,5-diaminopentane or 1,2-diaminopentane, 1,5-diamino-2-methylpentane, diaminohexane, for example 1,6-diaminohexane or 1,2-diaminohexane, diaminoheptane, for example 1,7-diaminoheptane or 1,2-diaminoheptane, diaminooctane, for example 1,8-diaminooctane or 1,2-diaminooctane, diaminononane, for example 1,9-diaminononane or 1,2-diaminononane, diaminodecane, for example 1,10-diaminodecane or 1,2-diaminodecane, diaminoundecane, for example 1,11-diaminoundecane or 1,2-diaminoundecane, diaminododecane, for example 1,12-diaminododecane or 1,2-diamino-dodecane, 3,3′-dimethyl-4,4′-diaminodicyclohexylmethane, 4,4′-diaminodicyclohexylmethane, isophoronediamine, 2,2-dimethylpropane-1,3-diamine, 4,7,10-trioxatridecane-1,13-diamine, 4,9-dioxadodecane-1,12-diamine, polyetheramines, piperazine, 3-(cyclohexylamino)propyl-amine, 3-(methylamino)propylamine, N,N-bis(3-aminopropyl)methylamine.

Suitable aliphatic diols are linear, branched or cyclic aliphatic diols. Aliphatic diols which are suitable for crosslinking of the second mode are ethylene glycol, 1,2-propylene glycol, 1,3-propylene glycol, 2-methyl-1,3-propanediol, butanediols, for example 1,4-butylene glycol or butane-2,3-diol or 1,2-butylene gylcol, pentanediols, for example neopentyl glycol or 1,5-pentanediol or 1,2-pentanediol, hexanediols, for example 1,6-hexanediol or 1,2-hexanediol, heptanediols, for example 1,7-heptanediol or 1,2-heptanediol, octanediols, for example 1,8-octanediol or 1,2-octanediol, nonanediols, for example 1,9-nonanediol or 1,2-nonanediol, decanediols, for example 1,10-decanediol or 1,2-decanediol, undecanediols, for example 1,11-undecanediol or 1,2-undecanediol, dodecanediols, for example 1,12-dodecanediol, 1,2-dodecanediol, tridecanediols, for example 1,13-tridecanediol or 1,2-tridecanediol, tetradecanediols, for example 1,14-tetradecanediol or 1,2-tetradecanediol, pentadecanediols, for example 1,15-pentadecanediol or 1,2-pentadecanediol, hexadecanediols, for example 1,16-hexadecanediol or 1,2-hexadecanediol, heptadecanediols, for example 1,17-heptadecanediol or 1,2-heptadecanediol, octadecanediols, for example 1,18-octadecane-diol or 1,2-octadecanediol, 3,4-dimethyl-2,5-hexanediol, polyTHF, 1,4-bis(2-hydroxyethyl)-piperazine, diethanolamines, for example butyldiethanolamine or methyldiethanolamine.

Preferred polyalkylenepolyamines obtainable according to the invention comprise C₂-C₅₀-alkylene units, particularly preferably C₂-C₂₀-alkylene units. These can be linear or branched, they are preferably linear. Examples are ethylene, 1,3-propylene, 1,4-butylene, 1,5-pentylene, 1,2-pentylene and 1,6-hexylene, 1, 9-nonylene, 1,10-decylene, 1,12-dodecylene, 1,2-octylene, 1,2-nonylene, 1,2-decylene, 1,2-undecylene, 1,2-dodecylene, 1,2-tridecylene, 1,8-octylene, nonylene, decylene, undecylene, dodecylene, tridecylene, octyl, nonyl, decyl, undecyl, dodecyl, tridecyl, neopentylene. Cycloalkylene units are also possible, for example 1,3- or 1,4-cyclohexylene.

Compounds particularly suitable for the crosslinking of the second mode are those in which at least one of the aliphatic amino alcohols, aliphatic diamines or polyamines or aliphatic diols or polyols comprises an alkyl or alkylene group having from 2 to 4 carbon atoms.

Compounds particularly suitable for the crosslinking of the second mode are likewise those in which at least one of the aliphatic amino alcohols, aliphatic diamines or polyamines or aliphatic diols or polyols comprises an alkyl or alkylene group having five or more, preferably seven or more, particularly preferably nine or more, in particular twelve or more, carbon atoms.

Compounds particularly suitable for the crosslinking of the second mode are likewise those in which at least one of the starting materials aliphatic amino alcohols, aliphatic diamines or polyamines or aliphatic diols or polyols comprises an alkyl or alkylene group having from 5 to 50, preferably from 5 to 20, particularly preferably from 6 to 18, very particularly preferably from 7 to 16, especially preferably from 8 to 14 and in particular from 9 to 12 carbon atoms.

For the crosslinking of the second mode, preference is given to selecting at least (i) monoethanolamine or (ii) ethylene glycol with ethylenediamine. Furthermore, preferably at least ethylenediamine or 1,2-propylenediamine or 1,3-propylenediamine and 1,2-decanediol or 1,2-dodecanediol are preferably selected here.

It is also possible to use mixtures of aliphatic amino alcohols or mixtures of alkanediols or mixtures of diaminoalkanes in the respective reactions of the crosslinking of the second mode. The resulting polyalkylenepolyamines can comprise alkylene units of different length.

Polyfunctional amino alcohols having more than one OH group or more than one primary or secondary amino group can also be reacted with one another. In this case, highly branched products are obtained. Examples of polyfunctional amino alcohols are diethanolamine, N-(2-aminoethyl)ethanolamine, diisopropanolamine, diisononanolamine, diisodecanolamine, diisoundecanolamine, diisododecanolamine, diisotridecanolamine.

Polyols or mixtures of diols and polyols can also be reacted with diamines. Polyamines or mixtures of diamines and polyamines can also be reacted with diols. Polyols or mixtures of diols and polyols can also be reacted with polyamines or mixtures of diamines and polyamines. In this case, highly branched products are obtained. Examples of polyols are glycerol, trimethylolpropane, sorbitol, triethanolamine, triisopropanolamine. Examples of polyamines are diethylenetriamine, tris(aminoethyl)amine, triazine, 3-(2-aminoethylamino)propylamine, dipropylenetriamine, N,N′-bis(3-aminopropyl)ethylenediamine.

Hydroxyl and amino groups in diols, polyols and diamines, polyamines are, especially in the postcrosslinking of the second mode, preferably used in molar ratios of from 20:1 to 1:20, particularly preferably in ratios of from 8:1 to 1:8, in particular from 3:1 to 1:3.

In one embodiment of the process of the invention the water of reaction is removed using a suitable water separator.

In another embodiment of the process of the invention for increasing the molar mass, the water of reaction is removed by means of distillation, in which the water is stripped from the reaction system with or without addition of a suitable solvent (entrainer). The distillation in this case is preferably carried out continuously. Generally speaking, during the distillation, water may be the component having the lowest boiling temperature in the reaction mixture, and can therefore be removed from the system continuously or discontinuously. Furthermore, the water, as mentioned above, may be removed distillatively as an azeotrope with addition of a suitable solvent (entrainer).

In another embodiment of the process of the invention, the water of reaction is removed using an apparatus for phase separation. In this case, preferably, a portion of reaction mixture is led from the reactor continuously during the reaction, and is optionally cooled and run into one apparatus, or sequentially into two or more apparatuses, for phase separation, in which the water of reaction and the remainder of the reaction mixture undergo separation, and the water of reaction is removed from the system. With particular preference both phases are led separately from the apparatus for phase separation. With very particular preference the remainder of the reaction mixture here is returned to the reactor.

In a further embodiment of the process of the invention, the water is removed using a membrane.

In another embodiment of the process of the invention, the water of reaction is removed using a suitable absorber, as for example polyacrylic acid and salts thereof, sulfonated polystyrenes and salts thereof, activated carbons, montmorillonites, bentonites, and zeolites.

The various measures for removing the water of reaction can of course also be employed multiply and also in combination.

A homogeneous catalyst is understood as meaning a catalyst which is present in the reaction medium in homogeneously dissolved form during the reaction.

The homogeneous catalyst, which is used in the context of the process according to the invention for increasing the molar mass, generally comprises at least one element of the sub-groups of the Periodic Table of the Elements (transition metal). The alcohol amination can be carried out in the presence or absence of an additional solvent. The alcohol amination can be carried out in a multiphase, preferably one-phase or two-phase, liquid system at temperatures of generally 20 to 250° C. In the case of two-phase reaction systems, the upper phase can consist of a nonpolar solvent, which comprises the majority of the homogeneously dissolved catalyst, and the lower phase comprising the polar starting materials, the polyamines formed and also water. Furthermore, the lower phase can consist of water and also of the majority of the homogeneously dissolved catalyst, and the upper phase can consist of a nonpolar solvent which comprises the majority of the polyamines formed and the nonpolar starting materials.

In a preferred embodiment of the process of the invention, monoethanolamine is reacted in the presence of a catalyst, and with removal of the water formed during the reaction, through use of a water separator, an apparatus for distillative removal of water, one or more apparatuses for phase separation, or an absorbent.

In a further preferred embodiment of the process of the invention, diamines selected from ethylenediamine, 1,3-propylenediamine or 1,2-propylenediamine are reacted with dioles selected from ethylene glycol, 1,2-decanediol or 1,2-dodecanediol in the presence of a catalyst, and with removal of the water formed during the reaction, by use of a water separator, an apparatus for distillative removal of water, one or more apparatuses for phase separation, or an absorbent.

In another preferred embodiment of the invention, a polyalkylenepolyamine of relatively low molar mass is reacted in the presence of a catalyst to give a polyalkylenepolyamine with a higher molar mass, the polyalkylenepolyamine of relatively low molar mass having been prepared as described above in a preceding step from monoethanolamine or by reaction of ethylenediamine, 1,3-propylenediamine or 1,2-propylenediamine with ethylene glycol, 1,2-decanediol or 1,2-dodecanediol, and having been separated from the water of reaction.

The number of alkylene units n in the polyalkylenepolyamines is generally in the range of from 3 to 50 000.

The polyalkylenepolyamines thus obtained can carry both NH₂ and also OH groups as end groups at the chain ends.

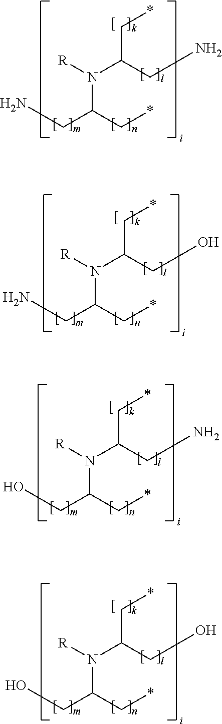

where preferably
R independently of one another, are identical or different and are H, C₁-C₅₀-alkyl,
l, m independently of one another, are identical or different and are an integer from the range from 1 to 50, preferably from 1 to 30, particularly preferably from 1 to 20,
n, k independently of one another, are identical or different and are an integer from the range from 0 to 50, preferably from 0 to 30, particularly preferably from 0 to 20,
i is an integer from the range from 3 to 50 000.

The number-average molecular weight Mn of the polyalkylenepolyamines obtained is generally from 200 to 2 000 000, preferably from 400 to 750 000 and particularly preferably from 400 to 100 000. The molar mass distribution Mw/Mn is generally in the range from 1.2 to 20, preferably from 1.5-7.5. The cationic charge density (at pH 4-5) is generally in the range from 4 to 22 mequ/g of dry substance, preferably in the range from 6 to 18 mequ/g.

The polyethyleneimines obtained according to the process according to the invention can be present either in linear form or in branched or multi-branched form, and also have ring-shaped structural units.

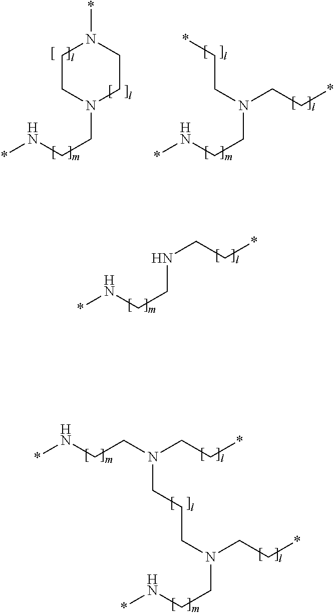

In this connection, the distribution of the structural units (linear, branched or cyclic) is random. The polyalkylenepolyamines thus obtained differ from the polyethyleneimines prepared from ethyleneimine by virtue of the OH end groups present and also optionally by virtue of different alkylene groups.

The catalyst is preferably a transition metal complex catalyst which comprises one or more different metals of the sub-groups of the Periodic Table of the Elements, preferably at least one element from groups 8, 9 and 10 of the Periodic Table of the Elements, particularly preferably ruthenium or iridium. The specified sub-group metals are present in the form of complex compounds. Numerous different ligands are contemplated.

Suitable ligands present in the transition metal complex compounds are, for example, phosphines substituted with alkyl or aryl, polydentate phosphines substituted with alkyl or aryl which are bridged via arylene or alkylene groups, nitrogen-heterocyclic carbenes, cyclopentanedienyl and pentamethylcyclopentadienyl, aryl, olefin ligands, hydride, halide, carboxylate, alkoxylate, carbonyl, hydroxide, trialkylamine, dialkylamine, monoalkylamine, nitrogen aromatics such as pyridine or pyrrolidine and polydentate amines. The organometallic complex can comprise one or more different specified ligands.

Preferred ligands are (monodentate) phosphines or (polydentate) polyphosphines, for example diphosphines, with at least one unbranched or branched, acyclic or cyclic, aliphatic, aromatic or araliphatic radical having 1 to 20, preferably 1 to 12 carbon atoms. Examples of branched cycloaliphatic and araliphatic radicals are —CH₂—C₆H₁₁ and —CH₂—C₆H₅. Suitable radicals which may be mentioned by way of example are: methyl, ethyl, 1-propyl, 2-propyl, 1-butyl, 2-butyl, 1-(2-methyl)propyl, 2-(2-methyl)propyl, 1-pentyl, 1-hexyl, 1-heptyl, 1-octyl, 1-nonyl, 1-decyl, 1-undecyl, 1-dodecyl, cyclopentenyl, cyclohexyl, cycloheptyl and cyclooctyl, methylcyclopentyl, methylcyclohexyl, 1-(2-methyl)pentyl, 1-(2-ethyl)hexyl, 1-(2-propylheptyl), adamantyl and norbornyl, phenyl, tolyl and xylyl, and 1-phenylpyrrole, 1-(2-methoxyphenyl)pyrrole, 1-(2,4,6-trimethylphenyl)imidazole and 1-phenylindole. The phosphine group can comprise two or three of the specified unbranched or branched, acyclic or cyclic, aliphatic, aromatic or araliphatic radicals. These may be identical or different.

Preferably, the homogeneous catalyst comprises a monodentate or polydentate phosphine ligand comprising an unbranched, acyclic or cyclic aliphatic radical having from 1 to 12 carbon atoms or an aryliphatic radical or adamantyl or 1-phenylpyrrole as radical.

In the specified unbranched or branched, acyclic or cyclic, aliphatic, aromatic or araliphatic radicals, individual carbon atoms can also be substituted by further phosphine groups. Also comprised are thus polydentate, for example bi- or tridentate, phosphine ligands, the phosphine groups of which are bridged by alkylene or arylene groups. The phosphine groups are preferably bridged by 1,2-phenylene, methylene, 1,2-ethylene, 1,2-dimethyl-1,2-ethylene, 1,3-propylene, 1,4-butylene and 1,5-propylene bridges.

Particularly suitable monodentate phosphine ligands are triphenylphosphine, tritolylphosphine, tri-n-butylphosphine, tri-n-octylphosphine, trimethylphosphine and triethylphosphine, and also di(1-adamantyl)-n-butylphosphine, di(1-adamantyl)benzylphosphine, 2-(dicyclohexylphosphino)-1-phenyl-1H-pyrrole, 2-(dicyclohexylphosphino)-1-(2,4,6-trimethylphenyl)-1H-imidazole, 2-(dicyclohexylphosphino)-1-phenylindole, 2-(di-tert-butylphosphino)-1-phenylindole, 2-(dicyclohexylphosphino)-1-(2-methoxyphenyI)-1H-pyrrole, 2-(di-tert-butylphosphino)-1-(2-methoxyphenyl)-1H-pyrrole and 2-(di-tert-butylphosphino)-1-phenyl-1H-pyrrole. Very particular preference is given to triphenylphosphine, tritolylphosphine, tri-n-butylphosphine, tri-n-octyl-phosphine, trimethylphosphine and triethylphosphine, and also di(1-adamantyl)-n-butyl-phosphine, 2-(dicyclohexylphosphino)-1-phenyl-1H-pyrrole and 2-(di-tert-butylphosphino)-1-phenyl-1H-pyrrole.

Particularly suitable polydentate phosphine ligands are bis(diphenylphosphino)methane, 1,2-bis(diphenylphosphino)ethane, 1,2-dimethyl-1,2-bis(diphenylphosphino)ethane, 1,2-bis(dicyclohexylphosphino)ethane, 1,2-bis(diethylphosphino)ethane, 1,3-bis(diphenyl-phosphino)propane, 1,4-bis(diphenylphosphino)butane, 2,3-bis(diphenylphosphino)butane, 1,3-bis(diphenylphosphino)propane, 1,1,1-tris(diphenylphosphinomethyl)ethane, 1,1′-bis-(diphenylphosphanyl)ferrocene and 4,5-bis(diphenylphosphino)-9,9-dimethylxanthene.

Furthermore, mention may preferably be made of nitrogen-heterocyclic carbenes, especially if, as described below, a polar solvent is added after the reaction, as particularly suitable ligands. In this connection, those ligands which form water-soluble complexes with Ru are very preferred. Particular preference is given to 1-butyl-3-methylimidazolin-2-ylidene, 1-ethyl-3-methylimidazolin-2-ylidene, 1-methylimidazolin-2-ylidene and dipropylimidazolin-2-ylidene.

Particularly suitable ligands which may be mentioned are also cyclopentadienyl and its derivatives mono- to pentasubstituted with alkyl, aryl and/or hydroxy, such as, for example, methylcyclopentadienyl, pentamethylcyclopentadienyl, tetraphenylhydroxycyclopentadienyl and pentaphenylcyclopentadienyl. Further particularly suitable ligands are indenyl and its derivatives substituted as described for cyclopentadienyl.

Likewise particularly suitable ligands are hydroxide, chloride, hydride and carbonyl.

The transition metal complex catalyst can of course comprise two or more different or identical ligands described above.

The homogeneous catalysts can be used either directly in their active form or else be produced starting from customary standard complexes such as, for example, [Ru(p-cymene)Cl₂]₂, [Ru(benzene)Cl₂]ₙ, [Ru(CO)₂Cl₂]ₙ, [Ru(CO)₃Cl₂]₂, [Ru(COD)(allyl)], [RuCl₃*H₂O], [Ru(acetylacetonate)₃], [Ru(DMSO)₄Cl₂], [Ru(PPh₃)₃(CO)(H)Cl], [Ru(PPh₃)₃(CO)Cl₂], [Ru(PPh₃)₃(CO)(H)₂], [Ru(PPh₃)₃Cl₂], [Ru(cyclopentadienyl)(PPh₃)₂Cl], [Ru(cyclopentadienyl)(CO)₂Cl], [Ru(cyclopentadienyl)(CO)₂H], [Ru(cyclopentadienyl)(CO)₂]₂, [Ru(pentamethylcyclopentadienyl)(CO)₂Cl], [Ru(pentamethylcyclopentadienyl)(CO)₂H], [Ru(pentamethylcyclopentadienyl)(CO)₂]₂, [Ru(indenyl)(CO)₂Cl], [Ru(indenyl)(CO)₂H], [Ru(indenyl)(CO)₂]₂, ruthenocene, [Ru(binap)Cl₂], [Ru(bipyridine)₂Cl₂*2H₂O], [Ru(COD)Cl₂]₂, [Ru(pentamethylcyclopentadienyl)(COD)Cl], [Ru₃(CO)₁₂], [Ru(tetraphenylhydroxy-cyclopentadienyl)(CO)₂H], [Ru(PMe₃)₄(H)₂], [Ru(PEt₃)₄(H)₂], [Ru(PnPr₃)₄(H)₂], [Ru(PnBu₃)₄(H)₂], [Ru(PnOctyl₃)₄(H)₂], [IrCl₃*H₂O], KIrCl₄, K₃IrCl₆, [Ir(COD)Cl]₂, [Ir(cyclooctene)₂Cl]₂, [Ir(ethene)₂Cl]₂, [Ir(cyclopentadienyl)Cl₂]₂, [Ir(pentamethylcyclopentadienyl)Cl₂]₂, [Ir(cyclopenta-dienyl)(CO)₂], [Ir(pentamethylcyclopentadienyl)(CO)₂], [Ir(PPh₃)₂(CO)(H)], [Ir(PPh₃)₂(CO)(Cl)], Dr(PPh₃)₃(Cl)] with the addition of the corresponding ligands, preferably the aforementioned mono- or polydentate phosphine ligands or the aforementioned nitrogen-heterocyclic carbenes, only under the reaction conditions.

The amount of the metal component in the catalyst, preferably ruthenium or iridium, is generally 0.1 to 5000 ppm by weight, in each case based on the total liquid reaction mixture.

The process according to the invention can be carried out either in a solvent or without solvent. The process according to the invention can of course also be carried out in a solvent mixture.

If the process according to the invention is carried out in a solvent, then the amount of solvent is often selected such that the polyalkylenepolyamines just dissolve in the solvent. In general, the weight ratio of the amount of solvent to the amount of polyalkylenepolyamines is from 100:1 to 0.1:1, preferably from 10:1 to 0.1:1.

Removal of the water of reaction during the reaction (synthesis of the polyalkylenepolyamine) may take place by means of the above-described measures, as for example with the aid of a water separator, by means of an apparatus for phase separation, by means of an apparatus for distillation or by means of a suitable absorber, either when the reaction is carried out with solvent, or when the reaction is carried out without solvent.

Removal of the water of reaction during the first or second postcrosslinking mode may likewise take place by means of the above-described measures, as for example with the aid of a water separator, by means of an apparatus for phase separation, by means of an apparatus for distillation or by means of a suitable absorber, either when the reaction is carried out with solvent or when the reaction is carried out without solvent.

Where the reaction or postcrosslinking is carried out without solvent, there is generally a phase present after the reaction or postcrosslinking that comprises the product and the catalyst. If the reaction or postcrosslinking is carried out with a solvent, this solvent generally has a higher boiling point than water, in the case of simultaneous distillative removal of the water from the reaction system. Suitable solvents are toluene or mesitylene, for example. Where during the reaction a solvent is used and one or more apparatuses for phase separation are used to remove the water, the boiling point of the solvent may be above or below the boiling point of water.

A first or second postcrosslinking mode of a polyalkylenepolyamine may be carried out both with and without solvent. Where the reaction is carried out without solvent, the homogeneous catalyst is in solution in the product, generally, after the reaction.

When the catalyst is in the product, it may remain in the product or may be removed therefrom by an appropriate method. Possibilities for the removal of the catalyst are, for example, wash removal with a solvent which is not miscible with the product, and in which the catalyst, as a result of a suitable choice of ligands, dissolves more effectively than in the product. The catalyst is optionally depleted from the product by means of multistage extraction. As extractant it is preferred to use a solvent which is also suitable for the target reaction and which, after concentration, can be used again for the reaction, together with the extracted catalyst. If the product is hydrophilic, then apolar solvents are suitable, such as toluene, benzene, xylenes, mesitylene, alkanes, such as hexanes, heptanes and octanes, and acyclic or cyclic ethers, such as diethyl ether and tetrahydrofuran. Additionally, alcohols having more than three C atoms, in which the OH group is bonded to a tertiary carbon atom, tert-amyl alcohol being an example, are suitable. If the product is lipophilic, then polar solvents are suitable, such as acetonitrile, sulfoxides such as dimethyl sulfoxide, formamides such as dimethylformamide, ionic liquids such as, for example, 1-ethyl-3-methylimidazolium hydrogensulfate, 1-butyl-3-methylimidazolium methanesulfonate. Also possible is the removal of the catalyst using a suitable absorber material.

Removal of the catalyst from a hydrophilic product after postcrosslinking or after a reaction in which water has been removed continuously may also take place by addition of water or an ionic liquid to the product phase, if the reaction is carried out in a solvent which is not miscible with water or with the ionic liquid. If, preferentially, the catalyst dissolves in the solvent used for the reaction, it can be removed from the hydrophilic product phase with the solvent, and optionally used again. This can be brought about by a choice of suitable ligands. The resulting aqueous polyalkylenepolyamines can be employed directly as technical polyalkylenepolyamine solutions. Removal of the catalyst from a lipophilic product after postcrosslinking or after a reaction in which water has been removed continuously may also be accomplished by addition of an apolar solvent to the product phase, if the reaction is carried out in a solvent which is immiscible with the apolar solvent—an ionic liquid, for example. If the catalyst here dissolves preferentially in the polar solvent, it can be removed from the apolar product phase with the solvent, and optionally used again. This can be brought about through a choice of suitable ligands.

If the postcrosslinking or reaction in which water is removed continuously is carried out in a solvent, this solvent may be miscible with the product and removed by distillation after the reaction. It is also possible to use solvents which exhibit a miscibility gap with the product or with the reactants. Suitable solvents for this purpose, in the case of hydrophilic products, include, for example, toluene, benzene, xylenes, mesitylene, alkanes, such as hexanes, heptanes and octanes, and acyclic or cyclic ethers, such as diethyl ether, tetrahydrofuran (THF), and dioxane, or alcohols having more than three C atoms, in which the OH group is bonded to a tertiary carbon atom. Preference is given to toluene, mesitylene, and tetrahydrofuran (THF), and also to tert-amyl alcohol. If the product is lipophilic, then suitability is possessed by polar solvents such as acetonitrile, sulfoxides such as dimethyl sulfoxide, formamides such as dimethylformamide, ionic liquids such as 1-ethyl-3-methylimidazolium hydrogensulfate, 1-butyl-3-methylimidazolium methanesulfonate, for example. As a result of a suitable choice of the ligands, the catalyst dissolves preferentially in the polar solvent phase.

The solvent can also be miscible under the reaction conditions with the starting materials and the product and only after cooling, for example to room temperature, form a second liquid phase which comprises the majority of the catalyst. Solvents which exhibit this property include, in the case of polar reactants and products, for example, toluene, benzene, xylenes, mesitylene, alkanes, such as hexanes, heptanes, and octanes. In the case of apolar products and reactants, ionic liquids, for example, exhibit these properties. The catalyst can then be separated off together with the solvent and be reused. The product phase can be admixed, in this variant as well, with water or with another solvent. The fraction of catalyst present in the product can then be separated off by suitable absorber materials such as, for example, polyacrylic acid and salts thereof, sulfonated polystyrenes and salts thereof, activated carbons, montmorillonites, bentonites and also zeolites, or else can be left in the product.

In the embodiment of the two-phase reaction regime, particularly suitable apolar solvents are toluene, benzene, xylenes, mesitylene, alkanes, such as hexanes, heptanes, and octanes, in combination with lipophilic phosphine ligands on the transition metal catalyst, such as triphenylphosphine, tritolylphosphine, tri-n-butylphosphine, tri-n-octylphosphine, trimethylphosphine, triethylphosphine, bis(diphenylphosphino)methane, 1,2-bis(diphenylphosphino)ethane, 1,2-dimethyl-1-,2-bis(diphenylphosphino)ethane, 1,2-bis(dicyclohexylphosphino)ethane, 1,2-bis(diethylphosphino)ethane, 1,3-bis(diphenylphosphino)propane, 1,4-bis(diphenylphosphino)butane, 2,3-bis(diphenylphosphino)butane and 1,1,1-tris(diphenylphosphinomethyl)ethane, and also di(1-adamantyl)-n-butylphosphine, 2-(dicyclohexylphosphino)-1-phenyl-1H-pyrrol and 2-(di-tert-butylphosphino)-1-phenyl-1 H-pyrrol, as a result of which the transition metal catalyst accumulates in the apolar phase. Suitable polar solvents include ionic liquids, dimethylsulfoxide and dimethylformamide, in combination with hydrophilic ligands on the transition metal catalyst, examples being nitrogen-heterocyclic carbenes, as a result of which the transition metal catalyst accumulates in the polar phase. In the case of this embodiment, in which the product and any unreacted reactants form a secondary phase enriched in these compounds, the majority of the catalyst can be separated off from the product phase by simple phase separation and be reused.

If volatile by-products or unreacted starting materials or else the water formed during the reaction or added after the reaction to improve extraction are undesired, these can be separated off from the product without problems by distillation.

The reaction according to the invention takes place in the liquid phase at a temperature of generally 20 to 250° C. Preferably, the temperature is at least 100° C. and preferably at most 200° C. The reaction can be carried out at a total pressure of from 0.1 to 25 MPa absolute, which may be either the intrinsic pressure of the solvent at the reaction temperature or else the pressure of a gas such as nitrogen, argon or hydrogen. The average reaction time is generally 15 minutes to 100 hours.

The addition of bases can have a positive effect on the product formation. Suitable bases which may be mentioned here are alkali metal hydroxides, alkaline earth metal hydroxides, alkali metal alcoholates, alkaline earth metal alcoholates, alkali metal carbonates and alkaline earth metal carbonates, of which 0.01 to 100 equivalents can be used based on the metal catalyst used.

The invention further provides polyalkylenepolyamines, in particular polyethyleneimines, which are prepared by the described embodiments of the process according to the invention.

A further subject of the invention are polyalkylenepolyamines which comprise hydroxyl groups, secondary amines or tertiary amines. The hydroxyl groups, secondary amines or tertiary amines are preferably located on a terminal carbon atom of an alkylene group, and therefore constitute an end group. These polyalkylenepolyamines preferably comprise hydroxyl groups.

For example, these polyalkylenepolyamines which comprise hydroxyl groups, secondary amines or tertiary amines are obtainable by means of the process of the invention. More particularly these polyalkylenepolyamines are obtained in one step in a process through the polymerization of monomers.

The ratio of the number of hydroxyl end groups to amino end groups (primary, secondary, tertiary) is preferably from 10:1 to 1:10, preferably from 5:1 to 1:5, more preferably from 2:1 to 1:2.

In a further preferred embodiment, polyalkylenepolyamines of this kind which comprise hydroxyl groups, secondary amines or tertiary amines comprise only hydroxyl end groups or only amine end groups (primary, secondary, tertiary). These polyalkylenepolyamines are preferably obtained by the process of the invention with the aid of a second postcrosslinking mode.

The invention, furthermore, also relates to the uses of these polyalkylenepolyamines a) as adhesion promoters for printing inks, b) as auxiliaries (adhesion) for producing composite films, c) as cohesion promoters for adhesives, d) as crosslinkers/curing agents for resins, e) as primers in paints, f) as wet-adhesion promoters in emulsion paints, g) as complexing agents and flocculating agents, h) as penetration assistants in wood preservation, i) as corrosion inhibitors, j) as immobilizing agents for proteins and enzymes, k) as curing agents for epoxide resins.

The present invention provides processes for increasing the molar mass of polyalkylenepolyamines in which no aziridine is used, no undesired co-products are formed and products of a desired chain length are obtained.

The invention is illustrated in more detail by the examples without the examples limiting the subject matter of the invention.

### EXAMPLES

The average molecular weight of the oligomers was determined by gel permeation chromatography in accordance with the method of size exclusion chromatography. The eluant used was hexafluoroisopropanol with 0.05% potassium trifluoroacetate. The measurement was carried out at 40° C. with a flow rate of 1 ml/min on a styrene-divinylbenzenecopolymer column (8 mm*30 cm) using an RI differential refractometer and/or UV photometer as detector. Calibration was carried out with narrow-range PMMA standards.

For the measurement of the Hazen color number (APHA method), the sample is diluted 1:2500 with a diluent which does not absorb in the range from 380 to 720 nm. The Hazen color number is then determined in a range from 380 to 720 nm, in 10 nm steps.

### Example 1

A 250 ml autoclave with paddle stirrer was charged under inert conditions, for the exclusion of oxygen, with 0.20 g (0.71 mmol) of [Ru(COD)Cl₂], 0.50 g (2.9 mmol) of 1-butyl-3-methylimidazolium chloride, 12.1 g (0.06 mol) of 1,2-dodecanediol, 20.0 g (0.27 mol) of 1,3-propylenediamine, 0.50 g (4.46 mmol) of potassium tert-butoxide, and 34 ml of toluene. The reaction mixture was stirred in the closed autoclave at 150° C. under the intrinsic pressure of the solvent for 20 hours. Following completed reaction and cooling, the reaction mixture was admixed with 5 ml of water and shaken, to give a solution (50.0 g) of the product in toluene, and also an aqueous solution (12.66 g) of the catalyst. The phases were separated and the catalyst phase was used again for example 2. From the product phase, the unreacted reactant and volatile constituents were removed on a rotary evaporator at 20 mbar and 120° C., giving 14.13 g of the pure product. The weight average (RI) of the oligomer obtained was 1470 g/mol, with. a dispersity (Mw/Mn) of 3.9. This corresponds to an average chain length n of the oligomer (CH₂CH(C₁₀H₂₁) NHCH₂CH₂NH)ₙ of 6. The color number was 74.

### Example 2

### First Postcrosslinking Mode

A 250 ml autoclave with paddle stirrer was charged under inert conditions with 0.20 g (0.71 mmol) of [Ru(COD)Cl₂], 0.50 g (2.9 mmol) of 1-butyl-3-methylimidazolium chloride, 0.50 g (4.46 mmol) of potassium tert-butoxide, 9.71 g of the discharge from example 1, and 34 ml of toluene. The reaction mixture was stirred in the closed autoclave at 140° C. under the intrinsic pressure of the solvent for 20 hours. Following completed reaction and cooling, the reaction mixture was admixed with 20 ml of water and shaken, to give a solution of the product in toluene, and also an aqueous solution of the catalyst. The phases were separated. From the product phase, the unreacted reactant and volatile constituents were removed on a rotary evaporator at 20 mbar and 120° C., giving 8.82 g of the pure product. The weight average (RI) of the oligomer obtained was 1740 g/mol, with a dispersity (Mw/Mn) of 3.7. This corresponds to an average chain length n of the oligomer (CH₂CH(C₁₀H₂₁) NHCH₂CH₂NH)ₙ of 7.3. For the measurement of the color number, the product was diluted 2500-fold in toluene. The color number was 200.

### Example 3

A 250 ml autoclave with paddle stirrer was charged under inert conditions with 12.1 g (7.63 mmol) of [Ru(PnOctyl₃)₄(H)₂], 450 g (7.37 mol) of ethanolamine, 10.05 g (89.56 mmol) of potassium tert-butoxide, and 1620 ml of toluene. In the closed autoclave, hydrogen was injected to 40 bar. The reaction mixture was then heated to 140° C. and stirred for 20 hours. After completed reaction and cooling, two phases formed. The upper phase, containing the catalyst, was separated on the lower phase, containing the product. The product phase was extracted by shaking with toluene. Thereafter the water of reaction, the unreacted reactant, and volatile constituents were removed on a rotary evaporator at 12 mbar and 116° C., giving 115.66 g of the pure product. The weight average (RI) of the oligomer obtained was 1470 g/mol, with a dispersity (Mw/Mn) of 2.8. This corresponds to an average chain length n of the oligomer (CH₂CH₂NH)ₙ of 34. The color number was 20.

### Example 4

### First Postcrosslinking Mode

A 250 ml autoclave with paddle stirrer was charged under inert conditions with 0.27 g (0.17 mmol) of [Ru(PnOctyl₃)₄(H)₂], 10.5 g of the discharge from example 3, 230 mg (2.05 mmol) of potassium tert-butoxide, and 37 ml of toluene. The reaction mixture was stirred in the closed autoclave at 140° C. under the intrinsic pressure of the solvent for 10 hours. After completed reaction and cooling, the product had precipitated as a solid. The batch was quenched with 200 ml of water, with the product dissolving and two phases being formed. The upper phase, containing the catalyst, was separated from the lower phase, containing the product. The water of reaction, the unreacted reactant, and volatile constituents were removed on a rotary evaporator at 12 mbar and 116° C., giving 9.42 g of the pure product. The weight average (RI) of the oligomer obtained was 1520 g/mol, with a dispersity (Mw/Mn) of 3.4. This corresponds to an average chain length n of the oligomer (CH₂CH₂NH)ₙ of 35. The color number was 71.

### Example 5

### First Postcrosslinking Mode

A 250 ml autoclave with paddle stirrer was charged under inert conditions with 0.27 g (0.17 mmol) of [Ru(PnOctyl₃)₄(H)₂], 10.5 g of the discharge from example 3, 230 mg (2.05 mmol) of potassium tert-butoxide, and 37 ml of toluene. In the closed autoclave, hydrogen was injected to 15 bar. Subsequently the reaction mixture was heated to 140° C. and stirred for 10 hours. After completed reaction and cooling, the product had precipitated as a solid. The batch was quenched with 200 ml of water, with the product dissolving and two phases being formed. The upper phase, containing the catalyst, was separated from the lower phase, containing the product. The water of reaction, the unreacted reactant, and volatile constituents were removed on a rotary evaporator at 12 mbar and 116° C., giving the pure product. The weight average (RI) of the oligomer obtained was 1170 g/mol, with a dispersity (Mw/Mn) of 3.4. This corresponds to an average chain length n of the oligomer (CH₂CH₂NH)ₙ of 27. The color number was 54.

### Example 6

### First Postcrosslinking Mode

A 250 ml autoclave with paddle stirrer was charged under inert conditions with 0.27 g (0.17 mmol) of [Ru(PnOctyl₃)₄(H)₂], 10 g of the discharge from example 3, 230 mg (2.05 mmol) of potassium tert-butoxide, and 37 ml of toluene. In the closed autoclave, hydrogen was injected to 20 bar. Subsequently the reaction mixture was heated to 150° C. and stirred for 10 hours. After completed reaction and cooling, the product had precipitated as a solid. The batch was quenched with 200 ml of water, with the product dissolving and two phases being formed. The upper phase, containing the catalyst, was separated from the lower phase, containing the product. The water of reaction, the unreacted reactant, and volatile constituents were removed on a rotary evaporator at 12 mbar and 116° C., giving 8.14 g of the pure product. The weight average (RI) of the oligomer obtained was 1550 g/mol, with a dispersity (Mw/Mn) of 3.3. This corresponds to an average chain length n of the oligomer (CH₂CH₂NH)ₙ of 36. The color number was 112.

### Example 7

### First Postcrosslinking Mode

A 250 ml autoclave with paddle stirrer was charged under inert conditions with 0.27 g (0.17 mmol) of [Ru(PnOctyl₃)₄(H)₂], 10 g of the discharge from example 3, 230 mg (2.05 mmol) of potassium tert-butoxide, and 37 ml of toluene. In the closed autoclave, hydrogen was injected to 40 bar. Subsequently the reaction mixture was heated to 160° C. and stirred for 5 hours. After completed reaction and cooling, the product had precipitated as a solid. The batch was quenched with 200 ml of water, with the product dissolving and two phases being formed. The upper phase, containing the catalyst, was separated from the lower phase, containing the product. The water of reaction, the unreacted reactant, and volatile constituents were removed on a rotary evaporator at 12 mbar and 116° C., giving 8.73 g of the pure product. The weight average (RI) of the oligomer obtained was 1460 g/mol, with a dispersity (Mw/Mn) of 3.3. This corresponds to an average chain length n of the oligomer (CH₂CH₂NH)ₙ of 34. The color number was 91.

1. A process for increasing the molar mass of a polyalkylenepolyamine by homogeneously catalyzed alcohol amination, comprising:
conducting a homogeneously catalyzed alcohol amination reaction of the polyalkylenepolyamine in a reactor with elimination of water in the presence of a homogeneous catalyst and
removing the water of reaction from the reactor; and
wherein the catalyst is a transition metal complex catalyst.

2. The process of claim 1, wherein, during the reaction, the polyalkylenepolyamine reacts with:
(i) an aliphatic amino alcohol; or
(ii) an aliphatic diamine or an aliphatic polyamine and an aliphatic diol or an aliphatic polyol.

3. The process of claim 2, wherein, during the reaction, the polyalkylenepolyamine reacts with (i) monoethanolamine.

4. The process of claim 1, wherein the water of reaction is removed during the reaction.

5. The process of claim 1, wherein the water of reaction is removed after the reaction.

6. The process of claim 1, wherein the water of reaction is removed continuously during the reaction.

7. The process of claim 1, wherein the catalyst comprises a monodentate or polydentate phosphine ligand.

8. The process of claim 1, wherein the catalyst comprises a nitrogen-heterocyclic carbene ligand.

9. The process of claim 1, wherein the catalyst comprises a ligand selected from the group consisting of cyclopentadienyl, substituted cyclopentadienyl, indenyl and substituted indenyl.

10. The process of claim 1, wherein the catalyst comprises a ligand selected from the group consisting of hydroxide, hydride, carbonyl and chloride.

11. The process of claim 1, wherein the reacting is carried out in the presence of a solvent or solvent mixture.

12. A polyalkylenepolyamine obtainable by the process of claim 1.

13. A polyethyleneimine obtainable by the process of claim 3.

14. An adhesion promoter for printing ink, an adhesion promoter in composite films, a cohesion promoter for adhesives, a crosslinker/curing agent for resins, a primer for paints, a wet-adhesion promoter for emulsion paints, a complexing agent, a flocculating agent, a penetration assistant in wood preservation, a corrosion inhibitor, an immobilizing agent for proteins and enzymes, or a curing agent for epoxide resins comprising the polyalkylenepolyamine of claim 12.

15. The process of claim 2, wherein, during the reaction, the polyalkylenepolyamine reacts with (ii) ethylene glycol with ethylenediamine.

16. An adhesion promoter for printing ink, an adhesion promoter in composite films, a cohesion promoter for adhesives, a crosslinker/curing agent for resins, a primer for paints, a wet-adhesion promoter for emulsion paints, a complexing agent, a flocculating agent, a penetration assistant in wood preservation, a corrosion inhibitor, an immobilizing agent for proteins and enzymes, or a curing agent for epoxide resins comprising the polyethyleneimine of claim 13.
